(19) United States
(12) Patent Application Publication
(10) Pub. No.: US 2026/0055447 A1
(43) Pub. Date: Feb. 26, 2026

(54) METHODS AND SYSTEMS FOR ABSOLUTE NUCLEIC ACID QUANTIFICATION

(71) Applicant: Yuchao Chen, Newark, CA (US)

(72) Inventor: Yuchao Chen, Newark, CA (US)

(73) Assignee: ARC Quantification Inc., Newark, CA (US)

(21) Appl. No.: 19/371,451

(22) Filed: Oct. 28, 2025

(51) Int. Cl. C12Q 1/6851

(52) U.S. Cl. CPC C12Q 1/6851

(57) ABSTRACT

The invention provides methods, systems, and reagent kits for absolute quantification of nucleic acids using a high-throughput, microfluidics-free workflow that integrates padlock-probe ligation, enzymatic cleanup, rolling-circle amplification, centrifugal deposition, and digital fluorescence imaging. Amplified products are deposited onto a planar multi-well plate and digitally enumerated by automated image analysis to determine absolute copy number without statistical partitioning or reference standards. The platform supports both DNA- and RNA-based workflows and multiplex detection using spectrally distinct or barcoded probes, enabling simultaneous quantification of multiple nucleic-acid targets with high precision, broad dynamic range, and low cost.

### FIELD OF THE INVENTION

The present invention relates generally to the field of nucleic acid analysis and quantification. More specifically, it concerns methods, systems, and reagent kits for absolute quantification of nucleic acids using a high-throughput, microfluidics-free workflow that integrates padlock-probe ligation, enzymatic cleanup, rolling-circle amplification, centrifugally deposition, and digital imaging-based enumeration.

### BACKGROUND OF THE INVENTION

Quantitative polymerase chain reaction (qPCR) is widely used for measuring nucleic acid abundance in research and diagnostics. However, qPCR quantifies target molecules indirectly by comparing fluorescence amplification curves against calibration standards, resulting in limited sensitivity, accuracy, and reproducibility.

To overcome these limitations, digital PCR (dPCR) was developed to provide absolute quantification of nucleic acids. In dPCR, a sample is partitioned into tens of thousands of individual micro-reactions—typically droplets, wells, or microchambers—such that each compartment statistically contains zero, one, or a few target nucleic acid molecules. After endpoint amplification, partitions are classified as positive or negative, and the absolute copy number is determined using Poisson statistics, eliminating the need for standard curves and reducing susceptibility to amplification efficiency variations.

While dPCR achieves higher precision and sensitivity, current systems rely heavily on microfluidic hardware and specialized consumables, leading to high cost, limited scalability, and complex operation. Typical dPCR platforms process up to 16-96 samples per run, with consumable costs of several dollars per sample and dynamic ranges constrained by the finite number of partitions.

Therefore, despite its technical advantages, current dPCR techniques suffer from key limitations that hinder broader adoption. These limitations include high instrumentation and consumable costs, low throughput, limited scalability, and a restricted dynamic range.

Additionally, reliance on microfluidic components and the Poisson distribution model adds complexity to manufacturing, end-user operation, and data analysis (e.g., rainfall patterns), often requiring specialized equipment and training. Given these challenges, there is a clear and growing need for alternative techniques that retain the core benefits of absolute quantification while improving on scalability, cost-efficiency, dynamic range, and ease of use.

A method that can eliminate the dependency on microfluidic systems, while enabling high-throughput analysis and a broader dynamic range, would represent a significant advance in nucleic acid quantification technology.

In parallel, rolling-circle amplification (RCA) has emerged as an isothermal alternative for nucleic acid detection. RCA amplifies circularized padlock probes to generate long, single-stranded DNA concatemers that can be fluorescently labeled and visualized. Early work by Lizardi et al. and Landegren et al. demonstrated padlock-probe ligation and RCA for mutation detection and signal amplification. This technique produces long, single-stranded DNA concatemers composed of repeating units complementary to the circular template, enabling signal amplification from extremely low amounts of starting material and eliminating the need for thermal cycling. RCA has been explored in various detection formats, including molecular imaging and flow cytometry, due to its capacity for signal enhancement and compatibility with direct visualization.

However, such RCA-based assays generally remained qualitative or semi-quantitative, limited by inefficient ligation, non-specific background, slow reaction kinetics, and difficulty in high-throughput visualization. RCA products are typically amplified and detected on microarrays, solid supports, or via in situ imaging formats, that exhibit limited reaction efficiency and scalability for absolute quantification. Therefore, there is a strong need to address these shortcomings while preserving the core advantages of RCA, such as isothermal amplification and high sensitivity.

Despite significant progress, no existing technology combines the precision of dPCR with the simplicity and scalability of RCA. dPCR systems require costly microfluidic partitioning and Poisson modeling, while RCA assays have not achieved reliable absolute quantification due to low enzymatic efficiency and inadequate detection formats. Furthermore, both approaches often depend on specialized instrumentation unsuited to routine or large-scale testing.

Accordingly, there is a continuing need for a microfluidics-free quantification platform that provides (i) true absolute quantification of nucleic acids without reliance on Poisson statistics, (ii) high scalability and high throughput using standard laboratory plates and centrifugation rather than droplet or chip partitioning, (iii) rapid isothermal amplification with high enzymatic fidelity and efficiency, and (iv) direct digital enumeration of amplification products by automated imaging analysis.

Unlike previous RCA or padlock-probe methods that rely on solid-phase amplification and detection (e.g., on microbeads or substrates) or require microfluidic compartmentalization, the present invention uniquely integrates centrifugal deposition of the entire reaction mixture with digital enumeration on standard multi-well plates, enabling rapid, high-throughput, scalable, and cost-effective absolute quantification.

Although widely adopted, real-time quantitative PCR (qPCR) lacks the ability to provide absolute quantification, leading to reduced precision. To overcome this limitation, digital PCR (dPCR) was developed, offering absolute quantification of nucleic acids with enhanced sensitivity and accuracy. However, its widespread use is challenged by high costs, limited scalability, narrow dynamic range, and operational complexity. As a result, there is a need for a new approach, which integrates ligation and rolling circle amplification (RCA) within a fundamentally reengineered workflow to enable true absolute quantification of nucleic acids.

The disclosed method addresses key limitations of both conventional dPCR and existing RCA-based methods. By eliminating the need for microfluidics, droplet generation, or specialized confinement systems, the method can be seamlessly implemented in standard laboratory environments, utilizing common equipment such as robotic liquid handlers and micro-well plates, to achieve automatic high-throughput and low-cost nucleic acid quantification. The present invention fulfills these needs.

### BRIEF SUMMARY OF THE INVENTION

The present invention provides methods, systems, and kits for absolute quantification of nucleic acids using a high-throughput, microfluidics-free workflow that integrates padlock-probe ligation, enzymatic cleanup, isothermal amplification, centrifugally deposition, and digital imaging-based enumeration. The invention enables accurate, cost-effective quantification of DNA or RNA molecules without droplet partitioning or Poisson modeling, thereby overcoming key limitations of existing digital PCR (dPCR) and rolling-circle amplification (RCA) platforms.

In one embodiment, a target nucleic acid is hybridized with a padlock probe and circularized by a thermostable DNA ligase under high-stringency conditions, followed by protease digestion of the ligase to prevent enzymatic interference. The circularized probe is then amplified through rolling-circle amplification using a phi29-family DNA polymerase.

In another embodiment, all RCA amplicons are centrifugally deposited onto a planar multi-well-plate substrate to spatially separate amplification products, which are subsequently imaged by fluorescence microscopy. Individual RCA products are digitally enumerated using an automated image-analysis algorithm to determine absolute copy number.

In the preferred embodiment, the platform achieves high throughput, broad dynamic range, and low cost while operating entirely on standard laboratory equipment.

In further embodiments, the invention encompasses multiplex and RNA quantification workflows, an integrated benchtop imaging and analysis system incorporating a line-scan time-delay-integration camera for rapid plate scanning, and software implementing machine-learning-based detection of single-molecule amplification signals. These embodiments collectively enable accurate, scalable, and automation-compatible absolute nucleic acid quantification suitable for clinical diagnostics, gene expression profiling, and research applications.

### SUMMARY

The present invention provides methods, systems, and kits for absolute quantification of nucleic acids using a high-throughput, microfluidics-free workflow. The invention addresses long-standing limitations of digital PCR platforms, including high cost, complex microfluidic partitioning, and limited dynamic range, by integrating a streamlined sequence of enzymatic and analytical steps that enable digital enumeration of nucleic acid molecules without physical compartmentalization.

In one aspect, the invention provides a method comprising: (i) hybridizing a padlock probe to a target nucleic acid; (ii) circularizing the probe with a high-fidelity thermostable DNA ligase under high-stringency conditions to improve ligation efficiency and sequence fidelity; (iii) enzymatically degrading residual ligase using a protease; (iv) amplifying the circularized probe through isothermal rolling-circle amplification (RCA) using a phi29-family DNA polymerase; (v) centrifugally depositing substantially all amplification products onto the planar surface of a multi-well plate; and (vi) imaging and digitally enumerating spatially resolved RCA amplicons to determine the absolute copy number of the target nucleic acid.

In another aspect, the invention provides an integrated system configured to perform the foregoing method. The system may include a reaction module for ligation and protease digestion, an amplification module comprising a phi29-family DNA polymerase, a deposition module configured to centrifugally transfer amplification products to a planar substrate, and an imaging and analysis module comprising a fluorescence microscope and an automated image-analysis processor trained to detect and enumerate individual amplification products. The system may further include a computer-readable medium storing instructions for image acquisition, feature extraction, and quantification of discrete amplification events.

In yet another aspect, the invention provides a reagent kit containing a thermostable ligase, a protease for ligase degradation, a DNA polymerase for RCA, and preformulated padlock probes and primers, together with instructions for performing centrifugal deposition and digital imaging for absolute quantification.

The disclosed methods and systems achieve accurate, cost-effective, and high-throughput absolute quantification across a broad dynamic range without requiring microfluidic chips, droplet generation, or Poisson distribution-based partitioning. By coupling high-fidelity enzymatic processing with spatially resolved digital imaging, the invention enables rapid quantification of DNA or RNA molecules using standard laboratory infrastructure, supporting applications in molecular diagnostics, gene expression analysis, infectious disease detection, and nucleic acid research.

### BRIEF DESCRIPTION OF THE DRAWINGS

The following drawings form part of the present specification and are included to further illustrate and explain certain embodiments and aspects of the present disclosure. The drawings, together with the detailed description, serve to explain the principles of the invention and its various features and advantages.

FIG. 1 illustrates a representative design of a padlock probe 100 comprising a primer-binding site 101, two fluorescent probe binding sites 102a and 102b, and two target-binding arms 103a and 103b located at the 3′ and 5′ ends for hybridization and circularization.

FIG. 2(a)-2(e) schematically depicts one embodiment of the assay workflow of the Absolute Rolling-Circle Quantification (ARC-Q) method, including:
(a) hybridization of the padlock probe 100 to the target nucleic acid 201;
(b) circularization of the probe 100 by thermostable ligation to generate circularized DNA template 210;
(c) enzymatic digestion of ligase by protease and hybridization of primer 221 to circularized DNA template 210;
(d) rolling-circle amplification of the circularized template 210 to produce an amplification product 231 labeled with fluorescent detection probes 232; and
(e) centrifugal deposition of amplification products 231 onto a multi-well-plate substrate 241 followed by imaging with a fluorescent microscope 242.

FIG. 3(a)-3(c) shows representative data of one embodiment comparing reaction efficiencies and optimization parameters, including:
(a) ligation efficiency of T4 DNA ligase versus thermostable HiFi Taq DNA ligase;
(b) rolling-circle amplification yield with and without protease treatment; and
(c) amplification yield obtained using wild-type phi29 DNA polymerase versus engineered phi29-XT polymerase.

FIG. 4 illustrates, in one embodiment, fluorescence imaging of individual rolling-circle amplification products deposited on a 384-well-plate substrate and digital enumeration of discrete amplification signals by automated image analysis.

FIG. 5(a)-5(c) compares, in one embodiment, quantitative results obtained by ARC-Q and by digital PCR (dPCR) across different target concentrations, showing representative fluorescence-microscopy images of individual amplification products.

FIG. 6(a)-6(b) illustrate two strategies for RNA quantification: FIG. (a) direct RNA 601-templated padlock-probe 100 ligation and FIG. (b) reverse-transcription-based ARC-Q workflow converting RNA 601 to cDNA 602 prior to ligation and amplification.

FIG. 7(a)-7(e) depict embodiments of the integrated ARC-Q imaging and analysis system. FIG. 7(a) shows the design of the fully integrated imaging system. FIG. 7(b) presents a photo of the prototype device. FIG. 7(c) shows a cross-sectional schematic of the imaging system. FIG. 7(d) provides a representative fluorescence image of RCA products acquired using a time-delay-integration (TDI) camera. FIG. 7(e) illustrates example image-analysis results from the trained CNN model, with mean detection confidence values labeled for each identified signal.

### DETAILED DESCRIPTION OF THE INVENTION

### Definitions

As used in this specification and the appended claims, the singular forms “a,” “an,” and “the” include plural references unless the context clearly dictates otherwise. For example, a reference to “a probe,” “a target,” or “a droplet” includes one or more such entities.

The term “associate” or “associating” means direct or indirect physical, chemical, or hybridization-based attachment. For example, a labeled detection probe may hybridize to a circularized padlock probe amplified by rolling-circle amplification (RCA), thereby associating a fluorescent signal with a target nucleic acid molecule.

The term “at least” followed by a number denotes the start of a range beginning with that number, and the term “at most” denotes the end of a range ending with that number, as commonly used in this field.

The term “complementarity” refers to the ability of a nucleic acid sequence to hybridize with another sequence through Watson-Crick or non-canonical base pairing. Percent complementarity denotes the proportion of residues capable of forming hydrogen bonds between two sequences. “Perfectly complementary” indicates full base pairing over the relevant region, whereas “substantially complementary” refers to at least 60%, 70%, 80%, 90%, 95%, 97%, 98%, 99%, or 100% complementarity over a defined sequence length (e.g., 10-50 nucleotides) or hybridization under stringent conditions.

The term “comprises” and grammatical equivalents thereof mean that additional elements or steps may be included. Thus, a method “comprising” steps of probe ligation and amplification may optionally include further steps such as washing, signal imaging, normalization, or computational analysis.

The term “hybridizing” refers to the binding or annealing of a nucleic acid to its complementary sequence under defined stringency conditions. “Stringent conditions” are those in which specific hybridization occurs preferentially to perfectly matched sequences, minimizing non-specific binding. Suitable hybridization and wash conditions are known in the art and may be adapted based on probe length, GC content, and desired discrimination efficiency.

The term “nucleic acid” (or “polynucleotide”) refers to a polymer of nucleotide bases, natural or synthetic, linked by phosphodiester or modified linkages, and may include modified bases, labels, or blocking groups. Nucleic acids may be single- or double-stranded DNA, RNA, or hybrids thereof.

The term “target nucleic acid” refers to a nucleic acid sequence to be detected or quantified using the ARC Quantification workflow. Targets may include genomic DNA, cDNA, RNA transcripts (e.g., mRNA, lncRNA, miRNA), or synthetic sequences. The target may originate from biological, clinical, or environmental samples.

The term “probe” refers to a designed oligonucleotide capable of recognizing, hybridizing to, and/or circularizing upon a target nucleic acid sequence. In ARC Quantification, probes may include padlock probes, bridge probes, or capture probes configured for ligation and rolling-circle amplification. Probes may optionally include labels, barcodes, or blocking groups.

The term “sample” refers to any material containing or suspected to contain nucleic acids of interest, including biological fluids, cell lysates, tissue extracts, or purified nucleic acids. Samples may be prepared manually or via automated workflows compatible with the ARC Quantification platform.

The term “sequentially” means that components, domains, or regions of a probe or nucleic acid are arranged in a defined 5′-to-3′ or 3′-to-5′ order. Non-functional flanking sequences or linkers may be present without affecting probe functionality.

The term “substrate” refers to a solid support, surface, or reaction platform (e.g., multi-well plate, microarray slide, or imaging chip) upon which amplification products are immobilized or detected. Substrates may be patterned, unpatterned, coated, or uncoated depending on the optical and chemical requirements of the assay.

### Methods

The following description is provided to enable any person skilled in the art to make and use the invention and sets forth the best mode contemplated by the inventors for carrying out the invention. Various modifications, equivalents, and alternatives will be apparent to those skilled in the art without departing from the spirit and scope of the invention as defined by the appended claims.

Conventional rolling-circle amplification (RCA) methods have struggled to achieve absolute quantification of nucleic acids due to several inherent limitations. A key bottleneck is the low ligation efficiency of commonly used T4 DNA ligase, which typically achieves only 40-50 percent circularization under standard conditions. This inefficiency leaves a substantial portion of probes unligated, leading to undetected targets and reduced quantification accuracy. Additionally, T4 DNA ligase lacks high sequence specificity, increasing the risk of off-target ligation and false-positive signals.

In one embodiment, the invention relates to methods, systems, and reagent kits for absolute quantification of nucleic acids using a high-throughput, microfluidics-free workflow. The method—referred to herein as Absolute Rolling-Circle Quantification (ARC-Q)—combines high-fidelity padlock-probe ligation, enzymatic cleanup, isothermal RCA, centrifugal deposition, and digital imaging to determine nucleic-acid copy number without droplet partitioning, microfluidic device, or Poisson-based statistics.

In another embodiment, as illustrated schematically in FIG. 2, the workflow performs all enzymatic steps in homogeneous solution and subsequently transfers the entire reaction volume to a planar substrate for single-molecule imaging. Unlike conventional digital PCR (dPCR) platforms that rely on droplet or microchamber partitioning—often limited to 16-96 reactions per batch—this approach uses standard multi-well microplates and centrifugation-based deposition to spatially separate amplification products without specialized microfluidic equipment. Combined with rapid isothermal amplification and high-speed fluorescence imaging, the assay can complete the entire process within approximately 3.5 hours. The workflow thereby eliminates complex consumables while maintaining digital-level quantification accuracy and throughput compatible with multi-well microplates, such as 96- and 384-well formats.

In another embodiment, the method achieves a broad dynamic range, typically enabling quantification from fewer than 10 to more than 10⁶ target molecules per reaction. Unlike dPCR, which relies on sample partitioning and Poisson statistical modeling to infer copy number, ARC-Q directly counts amplification events without compartmentalization. The reliance on Poisson distribution in dPCR inherently limits precision at high target concentrations, where multiple molecules per partition become increasingly probable, leading to signal saturation and underestimation. In contrast, the present approach performs quantification in an open, unpartitioned reaction, allowing analysis of the entire reaction volume with no dead volume or sampling bias. Additionally, it eliminates the need for external reference curves or internal standards, reducing assay complexity and improving consistency across runs.

In another embodiment, the assay employs a high-fidelity thermostable ligase, such as HiFi Taq DNA ligase, which demonstrates exceptional thermal stability and maintains activity at temperatures up to 95° C. This allows high-stringency ligation conditions that minimize nonspecific interactions. HiFi Taq DNA ligase achieves greater than 95 percent ligation efficiency and superior specificity in distinguishing perfectly matched sequences from single-base mismatches at the ligation junction. These properties make it ideally suited for applications requiring high fidelity, including single-nucleotide polymorphism (SNP) detection, padlock probe-based assays, and precision molecular diagnostics.

In another embodiment, the ligation and amplification reactions are conducted entirely in homogeneous liquid-phase solution, rather than on solid supports such as beads or immobilized substrates. Traditional solid-phase RCA workflows often suffer from restricted diffusion, limited enzyme accessibility, and prolonged reaction times. By contrast, this liquid-phase environment enables more efficient molecular interactions, accelerating reaction kinetics. Following amplification, RCA amplicons are deposited onto the well substrate by centrifugation to permit spatially resolved imaging.

In another embodiment, the use of phi29-XT DNA polymerase, an engineered variant of the conventional phi29 enzyme, provides approximately two- to four-fold faster amplification rates compared to the wild-type enzyme. Combined with HiFi Taq DNA ligase, which supports rapid and high-specificity ligation at elevated temperatures, the complete reaction can be finished within approximately 3.5 hours. This integrated liquid-phase strategy significantly improves speed and throughput relative to conventional bead- or chip-based RCA systems.

In another embodiment, similar high-processivity DNA polymerases capable of strong strand-displacement activity under isothermal conditions may be substituted for phi29 or phi29-XT polymerase. Examples include Bst DNA polymerase, Vent (exo-) DNA polymerase, or other phi29-family and replicase enzymes exhibiting comparable fidelity and processivity. These alternatives may be advantageous for specific reaction temperatures, buffer compatibilities, or cost considerations while maintaining equivalent amplification performance.

In the preferred embodiment, the method offers a significant cost advantage over conventional dPCR platforms. By eliminating the need for microfluidic chips, droplet generators, or specialized consumables, the per-sample consumable cost is dramatically reduced. The workflow is compatible with standard laboratory infrastructure, using common 96- or 384-well plates, and achieves a total consumable cost of less than one U.S. dollar per sample. This makes the assay highly accessible and scalable for applications where cost per test is a critical barrier to implementation.

Although previous studies have explored molecular quantification using ligation-based RCA methods, they typically require reference curves due to limited reaction efficiency or incomplete sampling of the reaction volume, resulting in lower precision and sensitivity compared to dPCR. In another embodiment, the present assay was systematically redesigned and optimized across probe design, enzymatic steps, and imaging workflow to enable true absolute quantification without the need for calibration curves or internal standards.

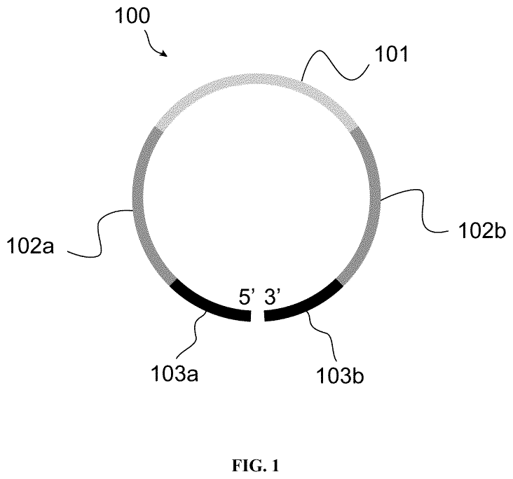

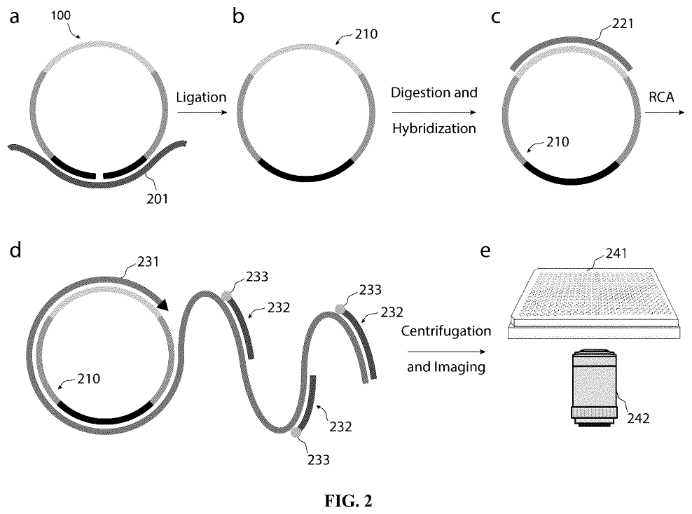

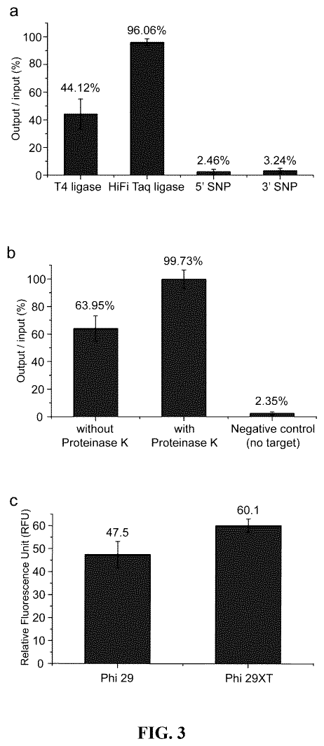

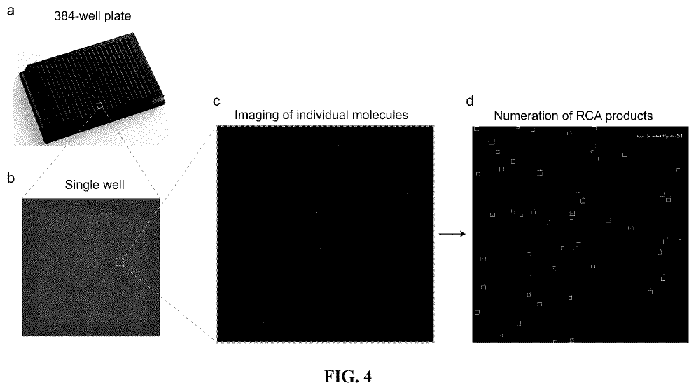

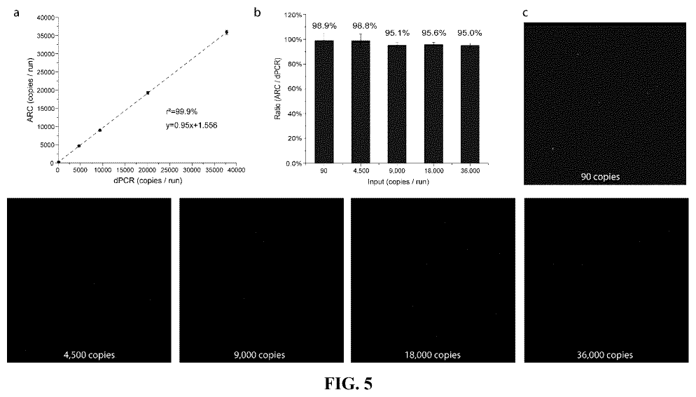

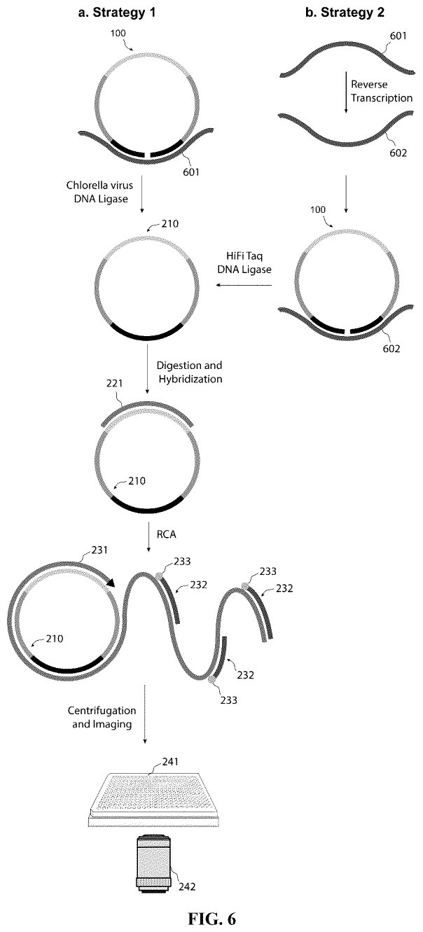

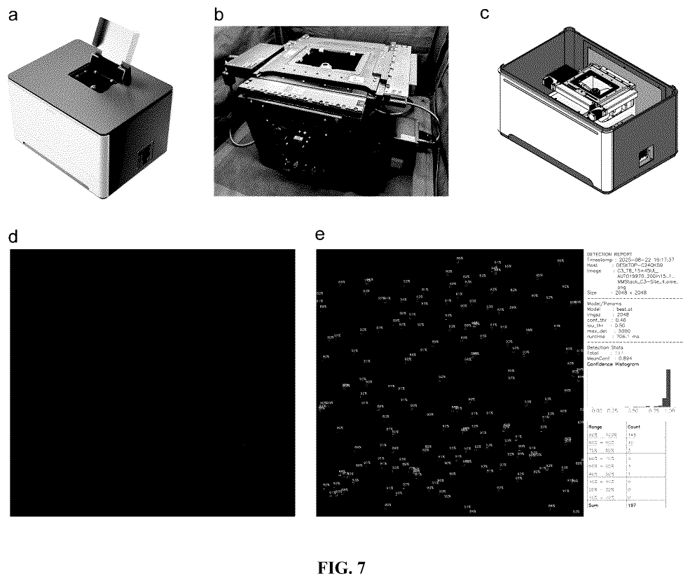

Padlock Probe. In one embodiment, as shown in FIG. 1, a padlock probe 100 is a linear oligonucleotide designed for specific target recognition and circularization to enable rolling-circle amplification (RCA). It contains two target-binding arms 103a and 103b, typically about 13 nucleotides in length, located at 5′ and 3′ ends, which hybridize to adjacent sequences on the target DNA, thereby positioning the probe ends for ligation.

In another embodiment, the central region of the probe includes one primer-binding site 101 (approximately 40 nucleotides) for RCA initiation and two internal fluorescent-probe recognition sequences 102a and 102b (approximately 20 nucleotides) for downstream detection. When hybridized, 3′ and 5′ ends of the probe 100 are juxtaposed on the target 201, permitting ligation into a circular template. With optimized sequence design, this probe provides high specificity, efficient amplification, and strong fluorescence signal intensity, enabling robust detection.

In another embodiment, the target-binding arm (103a and 103b) lengths may range from approximately 10 to 20 nucleotides, and the central region may include a primer-binding sequence 101 of about 30 to 60 nucleotides and internal probe sites of about 15 to 25 nucleotides.

In another embodiment, the probe 100 is synthesized with a 5′ phosphate group to facilitate ligation.

In certain embodiments, barcoded sequences or unique molecular identifiers are incorporated into the central region 102a and 102b to enable multiplex detection of multiple targets within a single reaction.

In some embodiments, the fluorescent detection-probe 232 sequence or dye 233 assignment is varied depending on the total number of targets in a multiplex assay, such that each target or barcode corresponds to a distinguishable fluorescent signal or emission wavelength.

As used herein, “nucleic acid” includes both deoxyribonucleic acid (DNA) and ribonucleic acid (RNA).

Hybridization. In one embodiment, as illustrated in FIG. 2(a), a target nucleic acid 201, which may be DNA or c-DNA derived from RNA, is combined with a padlock probe 100 in a hybridization buffer and heated briefly to denature secondary structures, followed by controlled cooling to permit specific annealing. In another embodiment, suitable buffer compositions may include Tris-HCl or similar buffering agents with salt concentrations between approximately 25 and 100 mM.

Ligation. In one embodiment, as illustrated in FIG. 2(b), to initiate the assay, padlock oligonucleotide probes 100 are mixed with target DNA 201 in the presence of HiFi Taq DNA ligase (New England Biolabs) or a functionally equivalent thermostable ligase and a corresponding ligation buffer. In one embodiment, the reaction mixture is first heated to approximately 90° C. for 1 minute to denature probes and target DNA, thereby facilitating efficient hybridization.

In another embodiment, the probe 100 ends are covalently joined by a high-fidelity thermostable DNA ligase, preferably HiFi Taq DNA ligase, under stringent conditions such as 37-45° C. for 20-60 minutes. These steps enable specific probe-target hybridization (FIG. 2(a)) and subsequent ligation into circular DNA templates 210 (FIG. 2(b)).

In another embodiment, in contrast to the commonly used T4 DNA ligase, HiFi Taq DNA ligase provides superior thermal stability and sequence specificity, enabling faster, more accurate, and higher-efficiency circularization of padlock probes 100. This enhanced ligation performance significantly improves the assay's overall sensitivity, specificity, and speed.

In the preferred embodiment, the high-fidelity thermostable DNA ligase, such as HiFi Taq DNA ligase or a functional equivalent, maintains catalytic activity at elevated temperatures and exhibits improved mismatch discrimination, providing high-fidelity circularization suitable for single-nucleotide-variant detection. In general, ligation efficiencies greater than 90 percent are achieved under these conditions, compared with less than 50 percent for conventional T4 DNA ligase.

Protease cleanup and primer hybridization. Following ligation, residual ligase may inhibit subsequent polymerase activity. In one embodiment, to eliminate inhibitory proteins, the reaction mixture is treated with a protease such as proteinase K (typically 0.5-2 U per 10 μL reaction) at 50-60° C. for 5-15 minutes, followed by thermal inactivation at 90-95° C.

In another embodiment, an RCA primer 221 is then annealed to the circularized probe (24). This thermal step promotes primer hybridization and denatures any remaining secondary structures, effectively preparing the reaction for robust and efficient rolling-circle amplification.

In the preferred embodiment, enzymatic cleanup markedly enhances RCA yield, typically from about two-thirds to near-quantitative conversion.

Rolling-circle amplification. In one embodiment, the circularized probe 210 serves as a template for isothermal amplification using a phi29-XT DNA polymerase or another phi29-family polymerase. The enzyme exhibits higher processivity and thermostability than wild-type phi29, allowing efficient amplification at approximately 40-45° C. for 1-3 hours in a reaction mixture containing deoxynucleotide triphosphates, an RCA primer 221, and one or more fluorescently labeled detection probes 232.

In another embodiment, the RCA primer 221 initiates synthesis by binding to the circular template 210 and displaces the hybridized target strand 201 through the strand-displacement activity of the polymerase, thereby producing long single-stranded RCA amplicons 231 containing hybridized fluorescent probes 232 for downstream detection.

In the preferred embodiment, the resulting single-stranded DNA concatemers 231 contain thousands of repeated sequence units complementary to the probe sequence 210.

Centrifugal deposition. In one embodiment, as shown in FIG. 2(e), upon completion of amplification, the entire reaction volume (for example, 20-50 μL) is transferred to a multi-well plate (241), typically a 384- or 96-well optical plate, and centrifuged at approximately 500-2,000×g for 5-15 minutes.

In another embodiment, this centrifugation step enables uniform deposition of RCA products onto the bottom surface of each well, forming a two-dimensional distribution of individual amplicons approximately 1-2 μm in diameter for downstream imaging.

In another embodiment, centrifugation deposits the RCA concatemers 231 uniformly onto the planar bottom surface 241, creating a sparse, two-dimensional layer suitable for optical imaging or scanning. Greater than 95 percent of the amplified products 231 are typically immobilized on the surface.

In the preferred embodiment, this planar configuration significantly reduces imaging time to approximately 15 seconds per well, resulting in about a 120-fold speed improvement compared to three-dimensional scanning, which typically requires around 30 minutes per sample.

In the preferred embodiment, the plate is imaged using a fluorescence microscope 242 equipped with a 10× objective lens and a scientific CMOS (sCMOS) camera (see FIG. 2(e)).

In another embodiment, the acquired images are analyzed using a deep learning-trained algorithm that detects and enumerates discrete RCA products 231, thereby enabling absolute quantification of target nucleic acids 201.

Fluorescence imaging and enumeration. In one embodiment, the deposited amplicons 231 are imaged using a fluorescence microscope 242 equipped with suitable excitation and emission optics and a scientific CMOS or CCD camera. Each RCA product 231 appears as a discrete bright fluorescent signal corresponding to an individual amplification event.

In another embodiment, automated image-analysis software counts these discrete signals directly, producing an absolute molecular count of the target nucleic acid 201. In certain embodiments, a convolutional neural network (CNN) or YOLO-type detector trained on annotated RCA images achieves detection precision above 95 percent.

In the preferred embodiment, enumeration is thereby performed without Poisson correction or statistical inference.

In another embodiment, substantially the entire amplification volume (for example, approximately 50 μL) is transferred to a standard 384-well plate and centrifuged at approximately 1000×g for 10 minutes. This step enables uniform deposition of RCA products 231 onto the planar bottom surface of each well 241, forming a two-dimensional distribution of individual amplicons approximately 1-2 μm in diameter for downstream imaging.

In the preferred embodiment, this planar configuration significantly reduces imaging time to approximately 15 seconds per well, representing about a 120-fold improvement in speed compared to three-dimensional volumetric scanning, which typically requires 30 minutes per sample.

In the preferred embodiment, the plate 241 is imaged using a fluorescence microscope 242 equipped with a 10× objective lens and an sCMOS camera (FIG. 2(e)). The acquired images are analyzed using a deep learning-trained algorithm that detects and enumerates discrete RCA products 231, thereby enabling absolute quantification of target nucleic acids 201.

Optimization studies. In one embodiment, as depicted in FIG. 3(a), the use of a thermostable ligase under stringent conditions significantly improves circularization yield relative to mesophilic enzymes. FIG. 3(b) shows that protease treatment following ligation further increases RCA efficiency. FIG. 3(c) demonstrates that the use of an engineered phi29 polymerase variant yields higher product fluorescence intensity than the wild-type polymerase. Collectively, these optimizations contribute to reproducible single-molecule quantification accuracy across a broad dynamic range.

In another embodiment, the key enzymatic steps involved in ligation and amplification were systematically compared, as shown in FIG. 3, to identify the optimal enzyme combination for the assay. Ligation efficiency was first evaluated using either T4 DNA ligase or a thermostable ligase (HiFi Taq DNA ligase). Ten reactions were conducted at 37° C. for 1 hour using optimized probes and primers with a synthetic double-stranded DNA target (82 bp) ranging from 0.01 to 20 amol (6×10³ to 1.2×10⁷ copies), followed by proteinase K digestion and RCA amplification for imaging. As shown in FIG. 3(a), HiFi Taq ligase consistently produced higher RCA yields (approximately 96%) compared to T4 ligase (approximately 44%) across all target concentrations, indicating the superior ligation efficiency of HiFi Taq ligase.

In another embodiment, HiFi Taq ligase was evaluated using DNA targets containing a single-nucleotide polymorphism (SNP) at either 5′ or 3′ end of the padlock probe. The background signal (approximately 3%) was comparable to that of the negative control, demonstrating the assay's high specificity for SNP discrimination.

In another embodiment, the effect of proteinase K treatment after ligation on RCA efficiency was assessed. As shown in FIG. 3(b), treatment with proteinase K (approximately 1.6 units for 10 minutes) followed by primer annealing significantly improved RCA yield (approximately 99%) compared to the untreated condition (approximately 64%). This result suggests that removal of residual ligase enhances polymerase activity during amplification.

In the preferred embodiment, the RCA yield was compared between wild-type phi29 DNA polymerase and an engineered variant, phi29-XT, using 5 fmol of input target DNA and a 2-hour incubation. As shown in FIG. 3(c), phi29-XT produced over 25% higher fluorescence signal (approximately 60 relative fluorescence units) than phi29 (approximately 48 relative fluorescence units), as measured by a fluorometric assay with YOYO dye labeling. This result highlights the superior amplification performance of phi29-XT, attributed to its enhanced efficiency and thermostability.

Together, these findings emphasize the importance of enzyme selection and post-ligation cleanup in maximizing the sensitivity, accuracy, and robustness of the assay.

Deposition and imaging performance. In one embodiment, referring to FIG. 4, centrifugation results in a uniform layer of amplification products on each well bottom. The resulting features are typically 1-2 μm in diameter and optically resolvable as distinct fluorescent puncta. Each well image may be assembled from multiple tiled frames, and automated enumeration can process hundreds of wells per hour. The system achieves plate-level throughput within less than 2 hours on standard microscopes and under 20 minutes on optimized line-scan systems.

In another embodiment, to enable high-throughput and sensitive detection of individual RCA products, a 384-well plate format compatible with fluorescence imaging was employed (FIG. 4a). Testing showed that over 99% of RCA products could be efficiently deposited onto the bottom surface of each well by centrifugation at 1000× g for 10 minutes, enabling spatial separation of amplified signals (FIG. 4b-c). Single-molecule fluorescence imaging revealed discrete spots corresponding to individual RCA amplicons, which were reliably visualized and quantified using a custom automated image analysis pipeline. This tool, based on the YOLOv8 object detection model, was trained on hundreds of annotated images to accurately identify RCA products. As illustrated in FIG. 4d, each detected signal is marked, allowing precise enumeration of RCA products per well. This setup combines the scalability of the 384-well format with single-molecule detection capabilities, offering a high-throughput and cost-efficient platform for digital nucleic acid quantification.

Quantitative benchmarking. In one embodiment, as shown in FIG. 5(a), ARC-Q exhibits linear correlation between counted signals and input target copies over at least five orders of magnitude. Recovery efficiencies exceed 90 percent at all tested concentrations. FIG. 5(b) illustrates agreement with commercial dPCR results, confirming equivalent accuracy while eliminating the need for microfluidic partitioning.

In another embodiment, to assess the quantitative accuracy of the ARC platform, we benchmarked its performance against dPCR using the QuantStudio Absolute Q Digital PCR System (ThermoFisher). As shown in FIG. 5a, copy numbers quantified by ARC exhibited a strong linear correlation with those obtained from dPCR across a dynamic input range of 90 to 36,000 copies, demonstrating high accuracy of the ARC method. Recovery efficiency was calculated as the ratio of copy numbers detected by ARC to those measured by dPCR. As shown in FIG. 5b, ARC consistently achieved >95% recovery across all tested concentrations, with the highest accuracy (98.9%) observed at 90 copies, revealing the platform's sensitivity and minimal target loss even at low input levels. Representative fluorescence images (FIG. 5c) further confirm these results, showing increased density of RCA amplicons corresponding to higher target inputs.

In the preferred embodiment, these results indicate the feasibility of the ARC platform as a robust method for absolute quantification of DNA.

RNA Quantification Strategies. In one embodiment, to extend the ARC Quantification (ARC-Q) platform beyond DNA detection, the invention provides two complementary strategies for RNA target 601 quantification, both leveraging the established rolling-circle amplification (RCA)-based workflow. The objective is to identify the approach that provides the highest accuracy, sensitivity, and operational simplicity for high-throughput RNA analysis while accommodating diverse RNA types and experimental conditions.

In one embodiment, a direct RNA-templated padlock ligation strategy is employed. As illustrated in FIG. 6(a), DNA padlock probes 100 are designed with termini that hybridize to adjacent regions of a specific RNA target sequence 601, enabling ligation without reverse transcription. The probes 100 may be synthesized with a 5′ phosphate group and hybridization arms of approximately 10-15 nucleotides, having melting temperatures between about 55° C. and 60° C. to ensure stable annealing at 37° C. Ligation is performed using Chlorella virus DNA Ligase, an enzyme exhibiting high efficiency and specificity for RNA-splinted DNA ligation. In one embodiment, the ligation reaction comprises approximately 2 nM padlock probe, 0.01-10 amol RNA target, and 1.25 U/μL Chlorella virus DNA Ligase in a 10 μL total volume, incubated at approximately 37° C. for 30-45 minutes.

In another embodiment, the circularized probes 100 are subsequently amplified by RCA using a phi29-XT DNA polymerase or other phi29-family polymerase with high processivity and thermostability, at about 42° C. for 1-3 hours, to generate concatemeric DNA products 231.

In another embodiment, the RCA products 231 are labeled with one or more fluorescent detection probes 232 (for example, Alexa 488-conjugated oligonucleotides) and imaged using the established ARC-Q plate-based format. Digital quantification is achieved by counting discrete RCA amplification signals per well using an automated image-analysis algorithm. This direct RNA-templated ligation bypasses reverse transcription, thereby minimizing bias arising from RNA secondary structures, chemical modifications, or limited transcript efficiency. Accordingly, it is particularly suitable for structured or small RNAs, such as microRNAs (miRNAs) and PIWI-interacting RNAs (piRNAs).

In another embodiment, a reverse-transcription-based ARC-Q workflow is employed, as illustrated in FIG. 6(b). In this approach, RNA targets 601 are first converted to complementary DNA (cDNA) 602 using a high-processivity reverse transcriptase, such as Maxima H Minus Reverse Transcriptase (Thermo Fisher Scientific), which exhibits strong tolerance for RNA secondary structures.

In another embodiment, the reverse-transcription reaction includes approximately 0.01-10 amol of synthetic RNA or 0.01-10 pg of total RNA, 300 nM gene-specific primers, and 10 U/μL enzyme in a 10 μL reaction volume, incubated at 50° C. for about 30 minutes, followed by heat inactivation at 85° C. for 5 minutes. The resulting cDNA 602 serves as the template for padlock-probe ligation using HiFi Taq DNA Ligase (New England Biolabs) in a 20 μL reaction containing 2 nM probe 100 and the derived cDNA 602, incubated at 39° C. for approximately 40 minutes, followed by RCA as described above.

In the preferred embodiment, both the direct RNA-templated and reverse-transcription-based strategies are validated in parallel using synthetic RNA oligonucleotides and total RNA extracted from HeLa cells. Comparative benchmarking is performed using digital PCR (dPCR) as the reference standard. Evaluation metrics include correlation (R²>0.99 versus dPCR), sensitivity (limit of detection <10 copies per reaction), reproducibility (coefficient of variation <10 percent across replicates), and specificity (cross-reactivity <1 percent). These complementary strategies collectively establish a validated ARC-Q workflow for RNA quantification that achieves performance metrics comparable to the DNA platform and enables applications in transcriptomic profiling, infectious-disease diagnostics, and RNA biomarker analysis.

Reverse-Transcription-Based ARC-Q Workflow. In another embodiment, the method involves converting RNA 601 to complementary DNA (cDNA) 602 prior to applying the ARC-Q workflow, thereby leveraging the greater chemical stability and uniformity of DNA intermediates. As illustrated in FIG. 6(b), RNA targets 601 are reverse-transcribed using a high-processivity reverse transcriptase, such as Maxima H Minus Reverse Transcriptase (Thermo Fisher Scientific), which exhibits strong tolerance for RNA secondary structures and displays optimal enzymatic activity at approximately 50° C.

In one embodiment, the reverse-transcription reaction includes approximately 0.01-10 amol of synthetic RNA or 0.01-10 pg of total RNA, 300 nM gene-specific primers, and 10 U/μL enzyme in a total reaction volume of 10 μL. The reaction is incubated at approximately 50° C. for 30 minutes, followed by heat inactivation at 85° C. for 5 minutes. The resulting cDNA 602 serves as the template for padlock-probe ligation using HiFi Taq DNA Ligase (New England Biolabs) or a functionally equivalent thermostable ligase.

In another embodiment, the padlock-probe ligation step is performed in a 20 μL reaction mixture containing approximately 2 nM probe 100 and the derived cDNA template 602, incubated at about 39° C. for 40 minutes, followed by rolling-circle amplification under the same conditions described in the first strategy. The RCA products 231 are subsequently labeled with fluorescent detection probes 232 and quantified through fluorescence imaging and digital enumeration of individual amplification events.

In another embodiment, this reverse-transcription-based method stabilizes the target sequence 601, thereby improving specificity and reproducibility for long or structured RNAs such as messenger RNA (mRNA) and long noncoding RNA (lncRNA). However, as will be understood by those skilled in the art, reverse transcription may introduce minor biases related to primer binding efficiency, RNA secondary structure, or enzyme processivity, which can affect quantification accuracy for certain RNA targets 601.

In the preferred embodiment, both the direct RNA-templated ligation strategy and the reverse-transcription-based workflow are evaluated in parallel using a panel of synthetic RNA oligonucleotides ranging from 20 to 100 nucleotides in length (Integrated DNA Technologies), including miR-21 (22 nt), piR-001102 (29 nt), and a 100-nt fragment of GAPDH mRNA, together with total RNA extracted from Hela cells using RNeasy Universal Kits (QIAGEN). RNA integrity is assessed and quantified using a Bioanalyzer (Agilent).

In another embodiment, digital PCR (dPCR), such as the Thermo Fisher Absolute Q platform, is used as the reference standard for performance benchmarking. Evaluation metrics include accuracy, measured as the correlation coefficient (R²) between ARC-Q and dPCR quantification across target concentrations; sensitivity, defined as the limit of detection (LOD); and reproducibility, evaluated as the coefficient of variation (CV) across triplicate measurements.

In another embodiment, comparative statistical analyses are performed using two-way analysis of variance (ANOVA) with post-hoc Tukey tests to evaluate differences between strategies and RNA types, with statistical significance defined as p<0.05. These studies yield a validated ARC-Q RNA quantification workflow with accuracy (R²>0.99 versus dPCR), sensitivity, and reproducibility comparable to the DNA-based ARC-Q platform. The optimal RNA quantification strategy for a given RNA class is selected based on its measured performance metrics and compatibility with target structure and sample type. Accordingly, both DNA- and RNA-based workflows share a common ligation-RCA-deposition-imaging architecture, enabling quantitative comparison within a single assay platform.

Multiplex Detection Workflow. In one embodiment, to extend the ARC Quantification (ARC-Q) platform from single-target to multiplex detection, the invention provides a workflow enabling simultaneous absolute quantification of multiple distinct nucleic-acid targets—such as DNA and RNA—within a single reaction. This approach leverages the high specificity of padlock probes 100 and the high fidelity of the thermostable ligases to uniquely tag each target 231 with a distinct sequence barcode. Each barcode is subsequently recognized by a sequence-specific fluorescent detection probe 232, enabling spectral separation of signals during high-throughput fluorescence imaging. In one embodiment, a four-plex ARC-Q configuration is implemented that maintains single-target accuracy, achieves a coefficient of variation (CV) below 10 percent, and exhibits no detectable cross-signal or false-positive detection.

Multiplex Detection. In some embodiments, the assay quantifies multiple targets per well by (i) assigning distinct fluorophores 233 to target-specific detection probes 232 (e.g., 2-6 channels); (ii) using combinatorial barcoding, wherein each padlock 100 includes a short barcode domain read out by one or more fluorescent “codebook” probes 232 to expand target count beyond the number of channels; and/or (iii) applying computational demixing to correct spectral bleed-through and classify signals.

Calibration includes imaging single-color controls to estimate a channel crosstalk matrix, followed by linear unmixing on spot-level intensities. Codebooks are designed with Hamming distance ≥2 to tolerate single-bit errors; decoding uses intensity thresholds or probabilistic classifiers. To reduce cross-hybridization, detection probes 232 are designed with Tm windows (+2-3° C.), GC 40-60%, and no 1° structure at assay temperature. Exemplary multiplexing quantifies 8-64 targets 201 and/or 601 per well with per-target precision ≥90% and recall ≥90% at typical spot densities (e.g., 10³-10⁵ RCA products/well).

In another embodiment, four representative nucleic-acid targets 201 and 601 are selected to evaluate multiplex performance. Target sequences are derived from known human transcripts, such as ACTB and GAPDH (mRNA), RNU6 (small nuclear RNA), and MALATI (long noncoding RNA). Each target is synthesized as both DNA 201 and RNA 601 oligonucleotides. For every target, a corresponding padlock probe 100 is designed with hybridization arms 101a and 101b of approximately 10-15 nucleotides and a unique 20-nucleotide barcode sequence 102a and 102b. Barcode sequences are computationally optimized using sequence-design software (for example, NUPACK) to minimize secondary-structure formation, avoid cross-hybridization (ΔG>−7 kcal/mol), and ensure compatibility with fluorescent probes 232 labeled with one or more spectrally distinct fluorophores 233, such as Alexa 405, Alexa 488, Alexa 568, and Alexa 647. This framework enables simultaneous detection of multiple targets 201 and/or 601 within a single multiplex assay while preserving quantitative accuracy.

In another embodiment, optimization of multiplex ARC-Q reactions focuses on minimizing cross-reactivity and ensuring uniform amplification efficiency across targets. Ligation conditions are systematically varied, including temperature between approximately 35° C. and 45° C., enzyme concentration between about 0.5 and 2 U/μL, and probe-to-target ratios ranging from 102:1 to 104:1, to prevent competitive inhibition among probes. In another embodiment, barcode-specific probe hybridization is optimized by adjusting annealing temperatures between approximately 50° C. and 65° C. and fluorescent-probe concentrations between about 5 nM and 20 nM to maximize signal-to-noise ratios greater than 5 while maintaining specificity. Cross-reactivity is evaluated by incubating padlock probes with non-target sequences at concentrations up to 100 nM, with false-positive signals constrained to less than 1 percent of the true target signal.

In another embodiment, validation of the multiplex ARC-Q workflow is conducted using mixtures of DNA and RNA targets 201 and 601 at equimolar ratios (1:1:1:1) and staggered ratios (for example, 1:10:100:1000) across a concentration range of approximately 1 zeptomole to 1 attomole. Total nucleic acids extracted from HeLa cells are used as a complex biological matrix to assess assay robustness under realistic sample conditions. In another embodiment, digital PCR (dPCR) serves as the reference method for benchmarking quantitative performance. Evaluation metrics include accuracy (correlation coefficient R²>0.99 versus dPCR), sensitivity (limit of detection defined by signal-to-noise ratio SNR >3), reproducibility (CV<10 percent across triplicates), and specificity (cross-reactivity <1 percent).

In the preferred embodiment, these studies establish a validated multiplex ARC-Q workflow capable of accurate, sensitive, and robust detection of multiple nucleic-acid targets in parallel within a single reaction. The multiplex platform expands the ARC-Q assay's throughput and analytical flexibility, enabling simultaneous profiling of different RNA or DNA analytes for use in research, diagnostic, and clinical monitoring applications.

These multiplex embodiments differ from conventional multi-color RCA assays by integrating computational unmixing and barcode decoding within the same digital enumeration pipeline, allowing expansion from ≤4 colors to ≥64 targets without adding detection channels.

Comprehensive Characterization of the ARC-Q Assay. In one embodiment, building on the optimized RNA and multiplex workflows described above, the invention provides a systematic and quantitative characterization of the ARC Quantification (ARC-Q) platform to define its analytical performance boundaries. The goal of this embodiment is to benchmark ARC-Q against digital polymerase chain reaction (dPCR) across multiple performance dimensions, including sensitivity, specificity, limit of detection (LOD), dynamic range, and throughput, thereby identifying the platform's strengths and operational limitations relative to established quantitative standards.

In another embodiment, a diverse target panel is assembled to assess performance across a range of molecular and structural contexts. The target panel includes synthetic DNA and RNA oligonucleotides of varying lengths (approximately 20-200 nucleotides), GC contents, and secondary-structure complexities, as well as total nucleic acids extracted from HeLa cells and heat-inactivated SARS-CoV-2 virus (ATCC) to represent complex biological samples. Each target is tested under both singleplex and four-plex conditions using the optimized workflows developed for DNA and RNA quantification. RNA measurements employ the optimal strategy identified for RNA ligation or reverse transcription, while DNA quantification follows the standard ARC-Q workflow.

In another embodiment, sensitivity and LOD are determined by serially diluting target nucleic acids across eight logarithmically spaced concentrations ranging from approximately 10⁷ to 10⁰ copies per reaction, with each dilution tested in triplicate. The LOD is defined as the lowest target concentration at which the assay produces quantifiable measurements with a coefficient of variation (CV) less than 15 percent across replicates. Assay specificity is evaluated using sequences containing single-nucleotide variations to confirm single-nucleotide-polymorphism (SNP)-level discrimination. Specificity is quantified as the ratio of cross-signal to target signal, with a threshold of less than 1 percent cross-reactivity.

In another embodiment, the dynamic range of ARC-Q is established by assessing the linearity of measured copy numbers across the dilution series using linear regression analysis against known concentrations, achieving a correlation coefficient (R²) greater than 0.99. Throughput is evaluated by scaling assay batch sizes and multiplex levels while measuring the total processing time from sample input to quantification. The workflow demonstrates the capability to process up to 384 samples per run within approximately five hours, including sample preparation and imaging, while maintaining accuracy and reproducibility.

In another embodiment, data analysis is conducted using two-way analysis of variance (ANOVA) with post-hoc Tukey tests to assess the effects of target type (DNA versus RNA) and assay configuration (singleplex versus multiplex) on each performance metric, with statistical significance defined as p<0.05. The results define the analytical parameters of the ARC-Q assay, including sensitivity, specificity, LOD, dynamic range, and throughput, and demonstrate quantitative performance directly comparable to dPCR.

In another embodiment, alternative configurations and potential optimization strategies are provided. (1) Because digital PCR typically exhibits a relatively narrow dynamic range (<10⁵ copies), high-concentration samples (>10⁶ copies) may be diluted prior to dPCR benchmarking to maintain quantifiable signal. (2) To ensure complete recovery of RCA products, centrifugation speed and duration may be optimized to achieve maximal deposition efficiency without product aggregation. (3) The assay's multi-step, one-pot enzymatic reactions may be further optimized by adjusting buffer pH, ionic strength, and cofactor concentrations to achieve optimal enzyme compatibility for both DNA and RNA workflows. (4) To minimize false-positive signals, particularly at low template concentrations, all assays are performed within an enclosed PCR workstation, with plate washing conducted by an automated washer and non-template negative controls included in each batch.

In the preferred embodiment, the fully characterized ARC-Q platform achieves a dynamic range spanning fewer than 10 to greater than 10⁶ copies per reaction, an LOD below 10 copies, an R² correlation above 0.99 when benchmarked against dPCR, and cross-reactivity below 1 percent for both single-nucleotide and multiplex detection. The assay achieves a throughput of up to 384 samples within five hours with a coefficient of variation below 15 percent for both singleplex and four-plex formats. Collectively, these results establish the ARC-Q platform as a validated, high-throughput, and broadly applicable system for absolute nucleic-acid quantification across DNA and RNA targets.

High-Speed Image Acquisition and Processing. In another embodiment, for large-scale operation, a custom high-speed imaging system employs a time-delay-integration (TDI) line-scan camera coupled to a telecentric optical system and motorized XY stage. The system synchronizes image capture with stage motion, producing distortion-free composite images at gigapixel-per-second rates. A 384-well plate can be imaged in approximately 10-20 minutes per fluorescence channel. Integrated flat-field correction and autofocus modules maintain consistent focus and illumination across wells.

In one embodiment, to increase imaging throughput for ARC-Q plate analysis, the invention provides an integrated benchtop device configured for automatic, high-speed sample scanning and fluorescence image acquisition. A prototype imaging module has been developed featuring a compact fluorescence microscope integrated with a scientific CMOS (sCMOS) camera, a motorized XY translation stage, a programmable microcontroller, and a multichannel illumination system, as illustrated in FIG. 7(b). Under software control, the system automatically positions the multi-well plate and acquires a series of fluorescence images—typically nine images per well—using a 10× objective lens. At this acquisition rate, the system captures one fluorescence channel across an entire 384-well plate in approximately 1.5 hours, establishing the baseline imaging speed for subsequent optimization.

In another embodiment, imaging speed is substantially increased by replacing the frame-based sCMOS camera with a time-delay-integration (TDI) camera, which operates as a high-sensitivity line-scan imaging device. The TDI sensor continuously collects photoelectrons across multiple pixel rows synchronized with the motion of the translation stage, thereby integrating signal during sample movement. Unlike conventional stepwise sCMOS imaging—which requires repeated acceleration, deceleration, and exposure at each field of view—the TDI approach enables continuous scanning across the entire well area, minimizing idle time and allowing real-time image acquisition during plate translation. This configuration provides markedly improved imaging throughput while preserving single-molecule detection sensitivity.

In another embodiment, TDI-based imaging is implemented using a high-resolution 9 K TDI camera having 256-pixel rows and 9 072 pixels per row, coupled with a precision piezo-driven XY stage capable of sub-micron positional accuracy and translation speeds up to approximately 750 mm/s. A telecentric optical system maintains constant magnification across the field of view, eliminating edge distortion during continuous scanning. Uniform excitation is provided by three high-power light-emitting-diode (LED) modules centered at approximately 390 nm, 465 nm, and a broadband emission peaking near 600 nm, together covering the spectral range of 360-750 nm. Rapid channel switching is achieved through electronic modulation with a transition time of less than 1 millisecond.

In another embodiment, multichannel imaging capability is achieved using a motorized filter-cube turret accommodating up to six fluorescence filter cubes, thereby supporting detection across as many as six distinct wavelength channels. Synchronization between stage translation and TDI image capture is maintained by hardware-triggered timing controllers, which precisely coordinate line-readout timing with stage motion to minimize motion blur and preserve spatial resolution. This synchronized design enables accurate single-molecule localization and quantitative signal enumeration during continuous plate scanning.

In the preferred embodiment, the fully integrated system provides high-throughput fluorescence imaging suitable for digital quantification of RCA products deposited in standard 96- or 384-well plates. The TDI-based architecture achieves an imaging throughput increase of more than ten-fold compared to conventional frame-based systems, enabling complete plate acquisition within approximately 15 minutes per fluorescence channel while maintaining quantitative precision equivalent to the slower sCMOS configuration.

In one embodiment, raw line-scan data generated by the time-delay-integration (TDI) camera are streamed directly to a high-speed acquisition board, such as a PCI Express (PCIe) Gen4 interface providing bandwidth greater than 5 gigabytes per second. The streamed data are assembled into two-dimensional images in real time using graphics-processing-unit (GPU)-accelerated stitching algorithms, which perform line registration, overlap blending, and frame compositing to reconstruct full-well fluorescence images with minimal latency.

In another embodiment, a prototype implementation using a Tucsen Dhyana 9K TDI camera demonstrated clear visualization of rolling-circle amplification (RCA) products with a high signal-to-noise ratio, as shown in FIG. 7(d). To ensure image fidelity and quantitative accuracy across the entire plate, the system incorporates multiple correction and stabilization subsystems, including (a) flat-field correction to compensate for illumination nonuniformity, (b) real-time background subtraction to enhance signal contrast, and (c) continuous focus stabilization using a near-infrared, laser-based autofocus module. The autofocus module maintains the focal plane alignment within submicron tolerance throughout plate scanning, compensating for plate warpage or thermal drift.

In another embodiment, performance modeling indicates that the TDI-based imaging system achieves line-acquisition rates up to approximately 510 kHz (equivalent to 4.59 gigapixels per second) with a read noise of less than two electrons per pixel. Under these conditions, an imaging area corresponding to approximately nine fields of view per well can be captured in less than two seconds. Consequently, a complete 384-well plate can be imaged within approximately 15 minutes per fluorescence channel, compared to greater than 1.5 hours using conventional frame-based imaging, representing a greater than sixfold increase in throughput.

In the preferred embodiment, completion of this subsystem yields an integrated benchtop imaging instrument, as illustrated in FIG. 7(a-c), combining TDI-based high-speed fluorescence scanning, precision motion control, and optimized telecentric optics. The resulting device establishes the hardware foundation for fully automated ARC Quantification at high-throughput plate scale, enabling single-molecule digital enumeration of nucleic acids across hundreds of samples per run with minimal operator intervention.

Automated Image Analysis and Deep-Learning Quantification. In one embodiment, to enable automatic detection and enumeration of single-molecule rolling-circle-amplification (RCA) products, the invention provides a computer-implemented image-analysis system comprising a deep-learning convolutional-neural-network (CNN) model trained for real-time recognition and quantification of fluorescence signals. Each fluorescent spot within the acquired images corresponds to an individual RCA amplicon. The analysis model automates identification, counting, and statistical evaluation of these discrete amplification events across entire multi-well plates.

In another embodiment, the system is implemented using a CNN-based object-detection framework such as YOLOv8, trained on hundreds of annotated fluorescence images collected with a conventional scientific CMOS (sCMOS) imaging system. The trained model demonstrates high sensitivity in distinguishing true RCA amplicons from background noise and optical artifacts. The network architecture employs multi-scale feature extraction and bounding-box regression to localize individual amplification products with single-pixel precision.

In another embodiment, because the upgraded imaging platform employs a time-delay-integration (TDI) camera rather than a frame-based sCMOS sensor, the image data exhibit differences in pixel geometry, line-acquisition profile, and intensity distribution. To accommodate these differences, a new training dataset is constructed by acquiring annotated images directly from the TDI imaging system. A hybrid dataset combining legacy sCMOS images and new TDI images is first used to evaluate model cross-compatibility. If variations in intensity scaling or background noise adversely affect detection performance, the CNN model is retrained exclusively on TDI-acquired images following preprocessing steps including line-stitching normalization, flat-field correction, and adaptive noise filtering to minimize sensor-specific artifacts.

In one embodiment, network training is performed locally on GPU-accelerated computing hardware using reconstructed two-dimensional images generated from the TDI line-scan data. Although the TDI camera acquires one line at a time, continuous line stitching during stage translation yields well-level images comparable in size (approximately 2048×2048 pixels) to those acquired by the prior sCMOS-based system, preserving the spatial resolution necessary for single-molecule detection. Data-augmentation techniques—including random rotation, scaling, illumination variation, and translation—are applied during training to enhance model robustness to imaging heterogeneity.

In another embodiment, initial testing using more than 300 annotated images demonstrates that the YOLOv8 model achieves a recall greater than 85 percent, a precision greater than 99 percent, and a mean average precision (mAP₅₀) exceeding 90 percent on validation datasets, as shown in FIG. 7(e). With an expanded training dataset comprising approximately 1 000 to 2 000 annotated TDI-stitched images, the model is expected to achieve recall above 95 percent and precision above 99 percent, providing highly reliable single-particle detection with minimal false-positive and false-negative results.

In the preferred embodiment, the deep-learning analysis pipeline operates in full compatibility with the high-speed TDI imaging system, reducing manual analysis time by more than 90 percent and increasing computational throughput by at least six-fold compared to prior workflows. The integrated platform ensures reproducible, unbiased ARC-Q quantification across 96- or 384-well plates, providing immediate digital enumeration of nucleic-acid amplification events following image acquisition.

In one embodiment, image data are processed in real time by GPU-accelerated software that extracts count, intensity, and positional metrics. Machine-learning models trained on labeled RCA datasets generate quality metrics such as confidence scores and density maps. Data can be processed locally or transmitted to a cloud server for automated reporting. Plate-to-plate reproducibility typically yields correlation coefficients greater than 0.99.

In certain embodiments, if the desired scanning speed is not achieved, a 1×2 pixel-binning mode of the TDI camera may be employed. This mode combines two adjacent pixels into one, resulting in a modest reduction in spatial resolution but decreasing exposure time by approximately 50 percent and improving the signal-to-noise ratio by about 1.5-fold, thereby further enhancing imaging efficiency.

In the preferred embodiment, completion of this subsystem yields an automatic, integrated benchtop system capable of scanning a full 384-well plate within approximately 15 minutes per fluorescence channel while maintaining detection precision ≥99 percent, recall ≥95 percent, and mean confidence ≥90 percent. The resulting system establishes a scalable, high-throughput analytical platform for absolute nucleic-acid quantification using the ARC-Q workflow.

Analytical and Clinical Validation of the ARC-Q Platform. In one embodiment, following completion of assay development and integration of the high-speed imaging and analysis subsystems described above, the invention provides methods for validating the analytical and clinical performance of the ARC Quantification (ARC-Q) platform across a variety of biological and clinical sample types. Conventional polymerase chain reaction (PCR) assays are highly sensitive to inhibitors—such as heme, polysaccharides, and humic acids—and typically require column- or bead-based nucleic-acid extraction procedures that can result in 30-50 percent recovery loss, particularly at low input concentrations. In contrast, the ARC-Q workflow, which employs padlock-probe ligation followed by rolling-circle amplification (RCA), exhibits greater tolerance to common PCR inhibitors. As a result, ARC-Q can be performed directly on minimally processed biological samples with limited pretreatment—such as heat, protease digestion, or dilution—to release nucleic acids and reduce inhibitory effects, without requiring complete extraction.

In another embodiment, this extraction-free workflow provides several advantages, including reduced sample loss, improved detection of short or fragmented nucleic acids, lower reagent cost, and shortened turnaround time. The invention therefore includes methods and systems configured to perform direct or extraction-free ARC-Q analysis on crude biological fluids while maintaining quantitative precision comparable to extraction-based methods. Comparative studies are conducted to evaluate extraction-free versus extraction-based workflows for a variety of primary biofluids and clinical matrices.

In another embodiment, a reagent kit for performing ARC-Q may comprise pre-measured reagents such as thermostable ligase, protease, engineered polymerase, padlock probes, primers, fluorescent detection probes, and instructions describing deposition and imaging steps. Optional consumables include pre-treated multi-well plates and calibration standards. The reagents may be formulated with stabilizers for extended shelf life under refrigerated or frozen storage.

In another embodiment, the assay tolerates moderate levels of potential inhibitors and can analyze minimally processed biofluids such as plasma, saliva, urine, or cerebrospinal fluid without extensive purification. Sample pretreatment may consist of brief protease digestion or dilution sufficient to release nucleic acids and reduce inhibitory effects. In certain implementations, clinically relevant targets such as viral RNA or circulating tumor DNA are detected with high sensitivity and specificity within approximately two to three hours from sample input to quantified result.

In another embodiment, the comparative data demonstrate that ARC-Q achieves accuracy equivalent to dPCR while providing greater dynamic range and reduced cost by eliminating droplet partitioning.

In another embodiment, a system configured to implement the described workflow may include a reaction module for ligation and protease digestion, an amplification module for RCA, a deposition module comprising a centrifugation unit or plate rotor, and an imaging and analysis module with fluorescence optics and embedded image-analysis software. These modules may be integrated into a benchtop instrument providing fully automated operation from ligation through data output.

In another embodiment, the ARC-Q platform provides absolute quantification without statistical partitioning, a dynamic range exceeding six orders of magnitude, high throughput of at least hundreds of samples per run, and low reagent cost. It is compatible with conventional laboratory automation and applicable to both DNA and RNA targets across clinical, environmental, and research settings.

The foregoing embodiments demonstrate a microfluidics-free strategy for precise nucleic-acid quantification combining high-fidelity ligation, enzymatic cleanup, isothermal amplification, centrifugal deposition, and digital imaging. Variations and modifications apparent to those skilled in the art may be made without departing from the scope of the invention as defined in the appended claims.

ARC-Q Validation in Primary Biofluids. In one embodiment, analytical validation of the ARC-Q assay is performed using pooled human plasma obtained from multiple donors. Synthetic double-stranded DNA oligonucleotides and single-stranded RNA mimics (Integrated DNA Technologies) targeting model oncogenic sequences, such as KRAS and EGFR hotspot regions, are spiked into the plasma at concentrations ranging from approximately 10 to 10⁶ copies per reaction in 20 μL aliquots. For each sample, two workflows are performed in parallel:
(a) extraction-based ARC-Q, using a commercial nucleic-acid extraction kit (for example, QIAamp, Qiagen), followed by ARC-Q detection; and
(b) extraction-free ARC-Q, employing minimal pretreatment of biofluid, such as heating, dilution, or proteinase digestion, to reduce viscosity and inhibitory effects.

In another embodiment, all ARC-Q assays are performed in triplicate, and quantitative results are obtained through enumeration of RCA amplicons (spots) using the deep-learning CNN-based object detector developed in Section [0121]-[0126]. Parallel aliquots of each sample are analyzed using digital PCR (dPCR) on the Thermo Fisher Absolute Q platform to serve as a reference method. Comparative performance metrics include:
Limit of Detection (LoD): defined as the lowest target concentration producing >95 percent positive replicates;
Limit of Quantification (LoQ): defined as the lowest concentration with coefficient of variation (CV)<20 percent;
Linearity: defined as correlation coefficient R²>0.95 across a five-log dynamic range;
Recovery: expressed as the percentage of input copies detected; and
Reproducibility: defined as intra- and inter-assay CV<15 percent across three independent runs.
   Statistical correlation between ARC-Q and dPCR is assessed using Deming regression, while Bland-Altman plots are used to evaluate measurement bias and 95 percent limits of agreement.

Validation Across Diverse Biofluids. In another embodiment, the ARC-Q assay is further validated across a broad panel of biofluids, including serum, urine, saliva, cerebrospinal fluid (CSF), and cell-culture medium. For each matrix, pooled samples from at least three donors are spiked with synthetic DNA and RNA targets at concentrations between approximately 10 and 10⁶ copies per reaction. Matrix-specific pretreatment conditions are determined through preliminary optimization, such as 1:5 dilution for urine to mitigate urea inhibition, brief centrifugation of saliva to remove cellular debris, and proteinase K digestion for CSF to degrade proteoglycans.

In another embodiment, both extraction-based and extraction-free ARC-Q workflows are evaluated in triplicate for each matrix. Quantitative performance is assessed using the same parameters defined above: sensitivity, linearity, reproducibility, and correlation with dPCR. Statistical analyses confirm assay robustness and reproducibility across all tested biofluids. These studies collectively establish the analytical validity of ARC-Q and demonstrate that extraction-free sample preparation yields comparable performance to conventional extraction-based methods, while substantially reducing processing time and material loss.

In the preferred embodiment, the optimized ARC-Q platform provides a universal nucleic-acid quantification system compatible with a wide variety of primary biofluids and minimally processed clinical samples. The method achieves reliable detection and quantification of nucleic acids directly from plasma, serum, urine, saliva, cerebrospinal fluid, or cell-culture media without microfluidic partitioning or extensive sample purification, establishing the foundation for clinical translation of the ARC-Q assay.

Validation of ARC-Q with Clinical Specimens. In one embodiment, the ARC Quantification (ARC-Q) platform is validated using clinically relevant specimens representing two major diagnostic contexts: (a) circulating cell-free DNA (cfDNA) in plasma for oncogenic variant detection and (b) viral RNA in saliva for infectious-disease testing. These studies demonstrate the platform's analytical sensitivity, specificity, and robustness in authentic clinical matrices.

cfDNA Mutation Detection. In one embodiment, the assay is applied to detection of single-nucleotide variants (SNVs) commonly used in precision oncology, including KRAS G12D (c.35G>A), EGFR L858R (c.2573T>G), EGFR T790M (c.2369C>T), and TP53 R175H (c.524G>A). Archived plasma samples obtained under Institutional Review Board-approved protocols from PrecisionMed and BioIVT include approximately 20 mutation-positive and 20 mutation-negative cases previously confirmed by next-generation sequencing (NGS). Each plasma specimen is divided into paired aliquots:
(a) a minimally pretreated aliquot analyzed directly by ARC-Q and digital PCR (dPCR); and
(b) an extraction-based aliquot processed by conventional silica-column or magnetic-bead nucleic-acid purification prior to ARC-Q and dPCR analysis.

In another embodiment, this paired design enables quantitative assessment of ARC-Q's capability to detect and quantify low-frequency variants within cfDNA. Performance metrics include:
Sensitivity, defined as the ability to detect variant allele fractions below 0.1 percent;
Specificity, defined as cross-reactivity with wild-type alleles below 0.1 percent; and
Quantification accuracy, expressed as correlation with dPCR results for variant and wild-type copy numbers.

Additionally, ARC-Q is evaluated for its capacity to retain and quantify short cfDNA fragments (<150 base pairs), which are frequently lost during extraction but are critical for early-stage disease detection.

In another embodiment, the ARC-Q workflow is validated for viral-RNA detection using inactivated SARS-CoV-2 (ATCC) spiked into pooled human saliva to generate approximately 40 contrived samples. The assay targets conserved viral genomic regions, including the N and ORFlab genes. Both extraction-based and extraction-free ARC-Q workflows are performed in parallel. Detection sensitivity is benchmarked using quantitative standards spanning 10 to 10⁵ genome equivalents per milliliter, and specificity is evaluated using non-SARS-CoV-2 control RNAs such as influenza A. These experiments define the analytical sensitivity, dynamic range, and cross-reactivity of the ARC-Q platform for pathogen detection in complex biological matrices.

In one embodiment, all clinical and contrived samples are analyzed in blinded fashion, with paired aliquots tested by both ARC-Q and dPCR under identical conditions. Robustness is further evaluated across pre-analytical variables, including multiple freeze-thaw cycles, short-term ambient storage, and exposure to common laboratory contaminants. All ARC-Q reactions are executed using an automated liquid-handling platform (for example, the Opentrons Flex Robot) equipped with integrated positive and non-template controls. Data acquisition and image analysis are performed through the high-speed TDI imaging system and deep-learning pipeline described in above, ensuring fully automated processing from sample to result.

In the preferred embodiment, completion of this validation establishes the ARC-Q platform as a clinically applicable system for accurate quantification of nucleic acids in plasma, saliva, and other biofluids. Benchmarking against dPCR demonstrates equivalent or superior analytical performance while providing a cost-efficient, extraction-optional workflow optimized for high-throughput testing. Target performance parameters include:
Limit of Detection (LoD)<50 copies per reaction;
Limit of Quantification (LoQ) with CV<15 percent for synthetic DNA/RNA standards;
Recovery >90 percent across multiple biofluids;
Sensitivity ≥95 percent and specificity ≥95 percent for clinically relevant cfDNA SNVs and SARS-CoV-2 RNA; and
Dynamic range 10-10⁵ genome equivalents per mL with linearity R²>0.95.
   Comparative analyses of extraction-free versus extraction-based workflows define the optimal strategy for each specimen type, confirming the robustness and versatility of ARC-Q across both research and clinical applications.

General Variations and Equivalents. In another embodiment, it will be understood by those skilled in the art that various substitutions, modifications, and functional equivalents may be employed without departing from the scope of the invention. For example, functionally similar DNA ligases, polymerases, reverse transcriptases, nucleases, or proteases may be substituted for the specific enzymes described herein, including thermostable, mesophilic, or engineered variants thereof. Alternative buffer compositions, cofactors, primers, probes, or reaction additives may be used to achieve comparable ligation, amplification, or detection performance. In certain embodiments, equivalent fluorescent dyes, quantum dots, or luminescent reporters may be substituted for the detection labels disclosed herein.

In another embodiment, comparable optical detection systems may be employed, including CCD, CMOS, sCMOS, EMCCD, or time-delay-integration (TDI) cameras, as well as alternative illumination sources such as lasers, LEDs, fiber-coupled lamps, or laser-scanning systems, with appropriate filters, objectives, or telecentric lenses optimized for specific wavelengths or resolution requirements. Equivalent sample supports, such as multi-well plates, chambered slides, coverslips, or microarray substrates, may be used in place of the illustrated plate formats. Comparable centrifugal, robotic, pneumatic, or microfluidic deposition systems may likewise be used to position or immobilize amplification products.

In another embodiment, comparable machine-learning or artificial-intelligence algorithms may be substituted for or combined with the disclosed models. Examples include YOLO-type detectors, convolutional neural networks (CNNs), U-Net architectures, transformer-based models, support-vector machines (SVMs), or ensemble-learning frameworks configured for fluorescence signal detection, segmentation, or classification. Equivalent computing architectures, including CPU-, GPU-, TPU-, or FPGA-based processors, or distributed cloud-computing systems, may execute analytical, image-processing, or quantification workflows. Data may be processed locally, on-premises, or within secure cloud environments for real-time or asynchronous reporting. In some embodiments, the invention may be implemented as a computer-readable medium containing program instructions for performing ARC-Q image analysis, digital quantification, and data visualization.

In another embodiment, comparable biofluids, tissues, or environmental matrices, including serum, plasma, saliva, urine, cerebrospinal fluid, tissue lysates, wastewater, or other biological extracts, may serve as analytical substrates. Equivalent synthetic nucleic acids, recombinant sequences, viral genomes, or plasmid standards may be employed for calibration or assay validation. The invention may also be adapted for use in clinical diagnostics, environmental monitoring, food-safety testing, agricultural genomics, biodefense, or industrial process control. Such variations, modifications, and substitutions are intended to be encompassed within the spirit and scope of the invention as defined by the appended claims.

Summary of the Invention and Advantages. In summary, the present invention provides an integrated, microfluidics-free platform for absolute nucleic-acid quantification based on padlock-probe ligation, enzymatic cleanup, isothermal rolling-circle amplification (RCA), centrifugal deposition, and digital fluorescence imaging. The workflow-referred to herein as Absolute Rolling-Circle Quantification (ARC-Q)-achieves single-molecule detection sensitivity, broad dynamic range, and high throughput using standard laboratory consumables without reliance on droplet or microchamber partitioning.

In one embodiment, the ARC-Q method employs a thermostable DNA ligase to achieve near-quantitative probe circularization, followed by protease-mediated enzyme inactivation and high-fidelity RCA using phi29-family polymerases. Centrifugal deposition of the amplified products onto the planar surface of a multi-well plate enables direct visualization and digital enumeration of discrete RCA amplicons by automated image analysis. The method provides absolute copy-number quantification across a dynamic range exceeding six orders of magnitude with reagent cost below one U.S. dollar per sample.

In another embodiment, the invention includes systems and apparatuses configured for high-speed, high-precision imaging of RCA products. A time-delay-integration (TDI) camera integrated with telecentric optics, motorized motion control, and synchronized multi-wavelength illumination achieves plate-scale imaging throughput greater than sixfold faster than conventional sCMOS systems. Raw line-scan data are processed in real time using GPU-accelerated reconstruction and analyzed through a deep-learning convolutional-neural-network (CNN) model trained to detect individual RCA signals with precision ≥99 percent and recall ≥95 percent.

In another embodiment, the ARC-Q platform supports both direct RNA-templated ligation and reverse-transcription-based workflows, providing accurate quantification of RNA targets without or with cDNA conversion. Multiplex detection is achieved by incorporating spectrally distinct barcode probes, enabling simultaneous measurement of multiple nucleic-acid species within a single reaction while maintaining cross-reactivity below one percent. Comprehensive validation demonstrates correlation coefficients (R²)>0.99 when benchmarked against digital PCR.

In the preferred embodiment, the ARC-Q workflow is validated for use with a wide range of biofluids and clinical specimens, including plasma, serum, urine, saliva, cerebrospinal fluid, and cell-culture medium, as well as for detection of viral RNA and circulating tumor DNA. The extraction-free protocol retains short and fragmented nucleic acids while reducing processing time and minimizing sample loss. The platform achieves LoD <50 copies per reaction, CV<15 percent, sensitivity and specificity ≥95 percent, and a dynamic range of 10-10⁶ copies, demonstrating equivalence or superiority to digital PCR across multiple performance metrics.

In one embodiment, Table 1 summarizes representative performance characteristics of the ARC Quantification (ARC-Q) platform compared to widely used commercial digital PCR instruments. As shown, ARC-Q provides higher throughput, broader dynamic range, faster processing time, and significantly lower per-sample cost while achieving equivalent quantitative accuracy.

TABLE 1

| | ARC | Bio-Rad | Thermo |
|---|---|---|---|
| | Quantification | QX200 | Absolute Q |
| Technique | Ligation- | ddPCR | dPCR |
| | based RCA | (Droplet) | (Microwell) |
| Throughput/run | 1-384 samples | 1-96 samples | 4-16 samples |
| Dynamic range | >6 logs | 4-5 logs | 4-5 logs |
| Unanalyzed | 0% (full- | 25-50% | ~5% |
| volume | volume | | |
| | analysis) | | |
| Speed | ~3.5 h for | ~7 hours for | ~1.5 h for |
| | 384 samples | 96 samples | 16 samples |
| Cost/sample | <$1.00 | $4.50-$8.00 | $10.00-$12.00 |

Collectively, these embodiments establish ARC-Q as a scalable, automated, and cost-efficient solution for digital nucleic-acid quantification. The system combines biochemical simplicity with advanced imaging and deep-learning analytics to deliver high-accuracy molecular counting at unprecedented throughput. By eliminating the need for microfluidic consumables, droplet generation, or partitioning, ARC-Q substantially reduces operational cost and complexity. The platform is applicable to diverse research, diagnostic, and clinical settings, including liquid biopsy, infectious-disease detection, gene-expression profiling, and molecular-diagnostic assay development, offering a transformative tool for high-throughput, quantitative analysis of nucleic acids.

What is claimed is:

1. A method for absolute digital quantification of nucleic acids, comprising:
(a) hybridizing one or more padlock probes to corresponding target nucleic acids in a sample;
(b) ligating the hybridized padlock probe with a DNA ligase to form a circularized template;
(c) treating the ligation reaction with a protease that degrades residual ligase and subsequently inactivating the protease;
(d) amplifying the circularized templates by rolling-circle amplification using a strand-displacing DNA polymerase with sufficient processivity to continuously synthesize concatemeric amplification products;
(e) transferring substantially the entire amplification reaction volume directly onto a planar substrate of a multi-well plate and centrifugally depositing amplification products onto the substrate surface to produce two-dimensionally separated discrete amplification signals;
(f) detecting and imaging each amplification product hybridized with one or more fluorescently labeled detection probes by fluorescence microscopy; and
(g) digitally enumerating the individual amplification events with an automated image-analysis processor to determine the absolute copy number of the target nucleic acids without requiring Poisson-based statistical inference or reference standards.

2. The method of claim 1, wherein the DNA ligase is a high-fidelity thermostable ligase, such as HiFi Taq DNA Ligase, or a functional equivalent enzyme exhibiting similar or improved thermostability and ligation fidelity under comparable reaction conditions.

3. The method of claim 2, wherein high-fidelity refers to the ability of the ligase to preferentially ligate perfectly matched padlock probe-target junctions while discriminating against single-base mismatches.

4. The method of claim 1, wherein the protease is proteinase K incubated between about 50° C. and 60° C., and thermally inactivated at or above 90° C.

5. The method of claim 1, wherein the polymerase is a phi29 DNA polymerase or an engineered phi29 polymerase exhibiting enhanced strand-displacement activity and processivity relative to the wild-type enzyme.

6. The method of claim 1, wherein the centrifugation of step (e) is performed under conditions sufficient to achieve uniform and reproducible deposition of amplification products, for example at a relative centrifugal force between 500×g and 2000×g for approximately 5 to 15 minutes.

7. The method of claim 1, wherein the substrate comprises an optical microplate compatible with automated fluorescence imaging, selected from 6-well, 12-well, 24-well, 48-well, 96-well, 192-well, 384-well, 1536-well formats, or combinations thereof.

8. The method of claim 1, wherein the digital enumeration is performed by a machine-learning algorithm trained to detect individual rolling-circle amplification products with at least about 90% recall and 90% precision.

9. The method of claim 1, wherein the assay provides a quantitative dynamic range of at least six orders of magnitude in a 96-well plate format and at least five orders of magnitude in a 384-well plate format, wherein the wells of each plate can be used either simultaneously or selectively for sample processing in a single run.

10. The method of claim 1, wherein the method is performed without droplet generation, microchambers, or microfluidic partitioning.

11. The method of claim 1, wherein the detecting comprises hybridizing target-specific fluorescent detection probes with mutually distinguishable emission spectra, imaging the amplification products in two or more fluorescence channels, and digitally enumerating each target based on channel-specific signal thresholds with spectral crosstalk compensation.

12. The method of claim 1, wherein each padlock probe comprises one or more internal sequence barcodes, and the detecting comprises hybridizing a set of fluorescent detection probes that specifically bind to the barcodes in predefined combinations across multiple fluorescence channels, thereby enabling multiplex target encoding.

13. The method of claim 12, wherein the detection probe may optionally be labeled with at least two fluorophores to enhance fluorescent signals.

14. The method of claim 12, wherein unbound fluorescent detection probes are optionally hybridized with complementary oligonucleotides labeled with corresponding quenchers to reduce background fluorescence and improve signal-to-noise ratio.

15. The method of claim 1, wherein the automated image-analysis processor performs spectral unmixing using a pre-measured crosstalk matrix and classifies individual amplification products to targets by maximum-likelihood or machine-learning assignment, thereby enabling simultaneous quantification of multiple distinct nucleic-acid targets within a single well.

16. The method of claim 1, wherein the target nucleic acid is DNA, and the ligation of step (b) is performed using a high-fidelity thermostable DNA ligase, such as HiFi Taq DNA Ligase, at a temperature of about 35° C. to 55° C. to circularize DNA padlock probes hybridized to DNA templates.

17. The method of claim 1, wherein the target nucleic acid is RNA, and the ligation of step (b) is performed using a Chlorella virus DNA ligase at a temperature of about 25° C. to 45° C. to circularize DNA padlock probes hybridized to RNA templates.

18. The method of claim 17, wherein the target RNA comprises one or more RNA molecules selected from any class of cellular or viral RNA, including messenger RNA, non-coding RNA, and synthetic RNA analogs.

19. The method of claim 1, wherein the target nucleic acid is RNA, and the method further comprises reverse-transcribing the target RNA into complementary DNA (cDNA) prior to padlock probe hybridization using a reverse transcriptase and primer.

20. A system for absolute nucleic acid quantification, comprising:
(a) a reaction module configured to circularize padlock probes using a high-fidelity thermostable ligase and to degrade the ligase with a protease;
(b) a phi29 DNA polymerase or phi29-family polymerase operative for isothermal rolling-circle amplification;
(c) a deposition module configured to centrifugally deposit substantially all amplification products onto a planar surface of a multi-well plate to spatially separate the amplification products; and
(d) an imaging and analysis module comprising a fluorescence microscope and an automated image-analysis processor configured to detect and enumerate individual amplification products to yield an absolute copy number of the target nucleic acid.

21. The system of claim 20, wherein the imaging and analysis module is configured for multiplex quantification of two or more nucleic-acid targets using distinct fluorescence channels, combinatorial barcodes, or computational unmixing.

22. The system of claim 20, wherein the modules are configured to automatically perform ligation, protease digestion, amplification, deposition, and imaging without microfluidic confinement.

23. A reagent kit for performing the method of claim 1, comprising:
(a) a high-fidelity thermostable DNA ligase;
(b) a protease for ligase degradation;
(c) a phi29 DNA polymerase or phi29-family polymerase operative for rolling-circle amplification;
(d) one or more padlock probes and primers, optionally including barcoded sequences for multiplex detection;
(e) one or more fluorescently labeled detection probes;
(f) a planar multi-well plate configured for centrifugal deposition of amplification products and compatible with fluorescence imaging; and
(g) instructions for performing centrifugal deposition and digital imaging quantification of the amplification products,
wherein the kit optionally further comprises Chlorella virus DNA ligase or reverse transcriptase and reverse-transcription primer for RNA quantification.
